(19) United States
(12) Patent Application Publication
(10) Pub. No.: US 2010/0129521 A1
(43) Pub. Date: May 27, 2010

(54) Heat Processed Products Having Altered Monomer Profiles and Processes For Controlling The Epimerization of (-)-Epicatechin and (+)-Catechin In The Products

(75) Inventors: John F. Hammerstone, JR., Easton, PA (US); Valeria Acquarone, Hackettstown, NJ (US)

Correspondence Address: GIBBONS P.C., ONE GATEWAY CENTER, NEWARK, NJ, 07102, US

(73) Assignee: MARS INC., MCLEAN, VA (US)

(21) Appl. No.: 11/993,546

(22) PCT Filed: Jun. 29, 2006
(86) PCT No.: PCT/US06/25423

§ 371 Date: Dec. 21, 2007

(51) Int. Cl. A23L 1/28

(52) U.S. Cl. 426/655; 426/520; 426/521

(57) ABSTRACT

A method for controlling the epimerization of (−)-epicatechin to (−)-catechin in an epicatechin-containing product, preferably an edible product, or of (+)-catechin to (+)-epicatechin in a catechin-containing product, comprises the step of heating the product at a temperature of up to about 200° C. and at a pH of up to about 8. Under either method, the epimerization may be carried out in an open food processor in a reduced oxygen atmosphere or in a closed food processor. The edible product may be pasteurized, boiled, or sterilized during the epimerization. Epimerization is minimized by lowering the heating temperature, by lowering the pH, and/or by lowering the heating time. Conversely, the epimerization is maximized by increasing the heating temperature, by increasing the pH, and/or by increasing the heating time. The edible product may contain or be a fruit product, a vegetable product, a cereal product, a bean product, a nut product, a spice product, or a botanical product.

### CROSS REFERENCE TO RELATED APPLICATIONS

This PCT application is a continuation-in-part of U.S. patent application Ser. No. 11/170,593 filed Jun. 29, 2005 for “Process for Controlling The Isomerization of (−)-Epicatechin and (+)-Catechin in Edible Food Products.”

### FIELD OF THE INVENTION

The invention is directed to novel products, particularly cocoa products, and to processes for controlling the epimerization of (−)-epicatechin to (−)-catechin and of (+)-catechin to (+)-epicatechin in edible products.

### BACKGROUND OF THE INVENTION

It is known that (+)-catechin and (−)-epicatechin undergo epimerization at the 2-position in a hot aqueous solution. The resulting epimers are (+)-epicatechin and (−)-catechin.

It is also known that epimers of tea catechins, which are primarily gallated monomers, are formed as the result of heat treatment. Reports on epimerization at the C-2 position undergone by (−)-epicatechin and (+)-catechin have not elucidated the role of pH on this reaction's kinetics nor the impact of pH on increasing or decreasing the rate of epicatechin to catechin epimerization. Most of the existing epimerization literature focuses on “tea catechins,” i.e., mostly gallated forms under heat treatment. Little or no emphasis has been placed on rigorously studying the reaction kinetics per se, nor on the factors that influence such kinetics.

Common food processing methods utilize heat at 72° C. (pasteurization), 100° C. and/or 125° C. (commercial sterilization) at a slightly acidic or neutral pH. It would be desirable to be able to control the level of epimerization of (−)-epicatechin and (+)-catechin in epicatechin- and catechin-containing food products, respectively, during the heat processing thereof, preferably under food conditions, to optimize the health benefits of the processed products.

### BRIEF SUMMARY OF THE INVENTION

The present invention provides a method for controlling the epimerization of (−)-epicatechin to (−)-catechin in an epicatechin-containing product by heating the product at a temperature of up to about 200° C. and at a pH of up to about 8. The present invention also provides a method for controlling the epimerization of (+)-catechin to (+)-epicatechin in a catechin-containing products by heating the product at a temperature of up to about 200° C. and at a pH of up to about 8. Preferably, the product is an edible product.

Isomerization of the naturally occurring epimers, (+)-catechin to (+)-epicatechin and (−)-epicatechin to (−)-catechin, respectively, is more correctly referred to as an epimerization. Epimerization is sometimes referred to as isomerization and the terms are used interchangeably herein. Epimers are a special type of diastereomer. They are a pair of stereoisomers with more than one chiral center which differs in chirality at one and only one chiral center. A chemical reaction which causes a change in chirality at only one of many chiral centers is referred to as an epimerization. Catechin and epicatechin have two chiral centers, one at the C-2 position and the other at the C-3 position. The changes that occur during the heating of products containing (+)-catechin and (−)-epicatechin occur only at the C-2 position.

In a preferred embodiment, the product has a water activity of about 0.2 to about 1.0. Also in a preferred embodiment, the temperature is about 72° C. to about 125° C., the pH is about 4 to about 7, and the time is at least about 15 seconds.

The epimerization may be carried out in an open food processor in a reduced oxygen atmosphere or in a closed food processor. Preferably, the epimerization is carried out in a modified or inert atmosphere. In this embodiment, the modified/inert atmosphere may be either under vacuum or under an inert gas. When an inert gas is used, the gas preferably is nitrogen, argon, or helium.

Depending on the temperature selected for the epimerization, the product may be either pasteurized or sterilized during the epimerization.

In one embodiment, the epimerization of (−)-epicatechin to (−)-catechin, or of (+)-catechin to (+)-epicatechin, may be minimized by lowering the heating temperature, by lowering the pH, and/or by lowering the heating time. In this embodiment, the temperature preferably is between about 37° C. and about 72° C., the pH preferably is between about 4 and about 6, and the time preferably is from about 15 seconds to about 30 minutes. In another embodiment minimizing epimerization of (−)-epicatechin to (−)-catechin, or of (+)-catechin to (+)-epicatechin, the temperature preferably is between about 37° C. and about 100° C., the time is preferably from about 1 second to about 1 hour for a pH greater than 6. In yet another embodiment minimizing epimerization of (−)-epicatechin to (−)-catechin, or of (+)-catechin to (+)-epicatechin, the temperature preferably is between about 72° C. and about 200° C., the time is preferably between about 1 second to about 1 hour for a pH less than or equal to 6. The process is particularly useful in a food product requiring heat pasteurization or sterilization.

Alternatively, the epimerization of (−)-epicatechin to (−)-catechin, or of (+)-catechin to (+)-epicatechin, may be maximized by increasing the heating temperature, by increasing the pH, and/or by increasing the heating time. In this embodiment, the temperature preferably is between about 100° C. and about 200° C., the pH preferably is between about 7 and about 8, and the time preferably is from about 1 minute to about 30 minutes. In another embodiment maximizing epimerization of (−)-epicatechin to (−)-catechin, or of (+)-catechin to (+)-epicatechin, the temperature preferably is between about 72° C. and about 200° C., the time preferably is between about 1 second to about 1 hour for a pH greater than or equal to 6. In yet another embodiment maximizing epimerization of (−)-epicatechin to (−)-catechin, or of (+)-catechin to (+)-epicatechin, the temperature preferably is between about 72° C. and about 200° C., the time preferably is about 1 hour or longer for a pH less than 6. The process is particularly useful in a food product requiring heat pasteurization or sterilization.

In the method for maximizing the epimerization of (−)-epicatechin to (−)-catechin, the epimerization preferably is carried out until an equilibrium mixture of about 70% (−)-catechin and 30% (−)-epicatechin is obtained. At equilibrium, the molar ratio of (−)-epicatechin to (−)catechin is 1:2. The same equilibrium point is favored for the epimerization of (+)-catechin to (+)-epicatechin, namely, the epimerization is carried out until an equilibrium mixture of about 70% (+)-catechin and 30% (+)-epicatechin is obtained, with a molar ratio of (+)-epicatechin to (+)-catechin of 1:2 following the epimerization.

Under either method, the product may contain or may be a fruit product, a vegetable product, a cereal product, a bean product, a nut product, a spice product, or a botanical product, or the extract thereof. The extract may be composed of flavanol monomers or proanthocyanidins, and preferably is composed of catechin, epicatechin and/or procyanidins. The preferred fruit products include blueberry, cranberry, blackberry, raspberry, strawberry, bilberry fruit, black currant, cherry, grape, apple, apricot, kiwi, mango, peach, pear and plum. The preferred vegetable product is Indian squash. The preferred cereal product is sorghum or barley. The preferred bean products include a black-eyed pea, a pinto bean, a small red bean, and a red kidney bean. The preferred nut product is an almond, a cashew, a hazelnut, a pecan, a walnut, a pistachio, or a peanut. The preferred spice product is a curry or cinnamon. The preferred botanical products include Chinese hawthorn, Acacia catechin, Pterocarpus marsupium, Cassia Nomane, rhubarb, rhodiola, pine bark, willow bark and Uncaria tomentosa (cat's claw).

In either method, the preferred food product is a cocoa product such as a food or beverage containing partially defatted or fully defatted cocoa solids, chocolate liquor, and/or a liquid or dry cocoa extract. Preferably, the food product is a dark chocolate bar, a dairy dessert, or a carbonated or milk beverage. Preferably, the cocoa solids, chocolate liquor and/or cocoa extracts are prepared from unfermented and/or underfermented cocoa beans. Preferably, the cocoa extract is comprised of catechin, epicatechin, and/or procyanidin oligomers thereof.

### BRIEF DESCRIPTION OF THE FIGURES

FIG. 1: Schematic diagram of a reaction apparatus for controlling epimerization of (−)-epicatechin to (−)-catechin or of (+)-catechin to (+)-epicatechin.

FIG. 2A: Graph of changes in concentration of (−)-epicatechin and (−) catechin over time, at a temperature of 72° C., pH 7.

FIG. 2B: Graph of changes in concentration of (−)-epicatechin and (−) catechin over time, at a temperature of 100° C., pH 6.

FIG. 2C: Graph of changes in concentration of (−)-epicatechin and (−) catechin over time, at a temperature of 100° C., pH 7.

FIG. 2D: Graph of changes in concentration of (−)-epicatechin and (−) catechin over time, at a temperature of 125° C., pH 4.

FIG. 2E: Graph of changes in concentration of (−)-epicatechin and (−) catechin over time, at a temperature of 125° C., pH 6.

FIG. 2F: Graph of changes in concentration of (−)-epicatechin and (−) catechin over time, at a temperature of 125° C., pH 7.

FIG. 3A: HPLC chromatograms showing time epimerization profiles, pH 7.4, 37° C., at 15, 30 and 60 minutes.

FIG. 3B: HPLC chromatograms showing time epimerization profiles, pH 7.4, 37° C., at 120 minutes, 180 minutes and 48 hours.

FIG. 4A: HPLC chromatograms showing epimerization profiles of epicatechin, water activity 0.2, 90% ethylene glycol, 10% water, pH 7.0: 30 seconds at 23° C., 1 minute at 37° C., 2 minutes at 62° C.

FIG. 4B: HPLC chromatograms showing epimerization profiles of epicatechin, water activity 0.2, 90% ethylene glycol, 10% water, pH 7.0: 2.5 minutes at 77° C., 3 minutes at 85° C., 3.5 minutes at 93° C.

FIG. 4C: HPLC chromatograms showing epimerization profiles of epicatechin, water activity 0.2, 90% ethylene glycol, 10% water, pH 7.0: 4 minutes at 100° C., 4.5 minutes at 108° C., 5 minutes at 116° C.

FIG. 4D: HPLC chromatograms showing epimerization profiles of epicatechin, water activity 0.2, 90% ethylene glycol, 10% water, pH 7.0: 6 minutes at 126° C., 7 minutes at 135° C., 8 minutes at 140° C.

FIG. 5: Graph of changes in concentration of (−)-epicatechin and (−) catechin over time, pH 7, water activity=0.2.

FIG. 6A: HPLC chromatograms showing time epimerization profiles, pH 7.0, 72° C., at 0, 5 and 10 minutes.

FIG. 6B: HPLC chromatograms showing time epimerization profiles, pH 7.0, 72° C., at 15, 20 and 25 minutes.

FIG. 6C: HPLC chromatograms showing time epimerization profiles, pH 7.0, 72° C., at 30, 40 and 50 minutes.

FIG. 6D: HPLC chromatograms showing time epimerization profiles, pH 7.0, 72° C., at 60, 75 and 90 minutes.

FIG. 6E: HPLC chromatograms showing time epimerization profiles, pH 7.0, 72° C., at 105, 120 and 180 minutes.

FIG. 6F: HPLC chromatograms showing time epimerization profiles, pH 7.0, 72° C., at 240, 300 and 360 minutes.

FIG. 7: HPLC chromatogram of catechin-epicatechin standard.

FIG. 8A: HPLC chromatogram showing epimerization of (−)-epicatechin to (−)-catechin in cocoa polyphenol extract, pH 3.8.

FIG. 8B: HPLC chromatogram showing epimerization of (−)-epicatechin to (−)-catechin in cocoa polyphenol extract, pH 7.0.

FIG. 9: Normal phase HPLC/FLD trace for the high CP cocoa powder.

FIG. 10: Normal phase HPLC/FLD data for the cooked high CP cocoa powder.

FIG. 11A to D: Normal phase HPLC/FLD data for high CP cocoa powder cooked for 30 min, 7.75 hours, and 24 hours.

FIG. 12: HPLC/FLD trace for the high CP cocoa extract.

FIG. 13: HPLC/FLD trace for the cooked high CP cocoa extract.

### DETAILED DESCRIPTION OF THE INVENTION

The present invention is directed to methods for controlling the epimerization of (−)-epicatechin to (−)-catechin and of (+)-catechin to (+)-epicatechin in products, preferably edible products, under most common food processing conditions, namely 72° C. (pasteurization), 100° C. or 125° C. (commercial sterilization) in a slightly acidic or neutral pH. As shown in the examples below, the rate and extent of epimerization can be controlled by varying the temperature and pH.

In the following examples, instantaneous temperature equilibration, which is necessary to accurately assess initial reaction rates, is achieved by performing the experiments in a thin tubular reactor immersed in a large thermostatic bath. Additionally, an inert atmosphere, which is necessary to avoid competitive loss of (−)-epicatechin by oxidation, is achieved by purging the pressurized feed tank containing reagent solution with nitrogen. While nitrogen is used in the following examples, it will be understood by those of ordinary skill in the relevant art that any inert gas, such as argon, may be used to achieve the same effect. Similarly, it will be understood that oxidation may be avoided by performing the epimerization reaction under vacuum. As a result, the epimerization reaction may be performed in an open food processor using a modified or inert environment (i.e., inert gas) or the reaction may be carried out in a closed food processor.

The following examples also demonstrate that the level of epimerization may be controlled as a function of temperature, pH, and reaction time. As shown in the following examples, the level of epimerization may be minimized by lowering the heating temperature, lowering the pH, and/or decreasing the heating time. Typically, the ingredients or products are heated at about pH 3.8 to about 7.0 and at about 37° to about 125° C. for about 1.0 minutes to several days. Preferably, they are heated at about pH 3.8 to about pH 6.0 at about 37° to about 100° C. for about 2 hours to several days. Most preferably they are heated at about pH 3.8 to about pH 5.0 and at about 37° to about 72° C. for about 2 days to several days. The level of epimerization may be minimized by lowering the pH, preferably by ≧0.2, more preferably by ≧0.4 and most preferably, by ≧1.0.

The level of epimerization may be minimized while minimizing the total heating process (i.e., minimizing the loss of CP and other detrimental effects on the product) at a pH≦6.0, preferably at a pH of about 3.8 to about 6.0, most preferably at a pH of about 3.8 to about 5.0; at a temperature of about 72° C. to about 200° C., preferably at about 85° C. to about 160° C., most preferably at about 100° C. to about 140° C.; for about 1 second to about 1 hour, more preferably for about 1 second to about 30 minutes, most preferably at about 1 second to about 15 minutes. Epimerization may be minimized while minimizing the total heating process effects at a pH>6.0, preferably at a pH of about 6.0 to about 7.0, most preferably at a pH of about 6.0 to about 6.5; at a temperature of about 37° C. to about 100° C., preferably at about 37° C. to about 90° C., most preferably at about 37° C. to about 80° C.; for about 1 second to about 1 hour, more preferably for about 1 second to about 30 minutes, most preferably at about 1 second to about 15 minutes.

Alternately, the level of epimerization may be maximized by increasing the heating temperature, increasing the pH (to a physiologic level, i.e., 7.4) and/or increasing the heating time, for a time and at a pH and temperature sufficient to epimerize the (−)-epicatechin. Typically, the ingredients or products are heated at about pH 3.8 to about 8 and at about 37° to about 200° C. for about 0.5 minutes to several days. Preferably, they are heated at about pH 5.0 to about pH 7.5 at about 72° to about 160° C. for about 1 minute to about 6 hours. Most preferably they are heated at about pH 6.0 to about pH 7.4 and at about 100° to about 140° C. for about 1.0 to about 4 hours. The level of epimerization may be maximized by raising the pH, preferably by ≧0.2, more preferably by ≧0.4 and most preferably, by ≧1.0.

The level of epimerization may be maximized while minimizing the total heating process (i.e., minimizing the loss of CP and other detrimental effects on the product) when performed at a pH of about ≧6.0, preferably at a pH of about 6.0 to about 8.0, most preferably at a pH of about 6.5 to about 8.0; at a temperature of about 72° C. to about 200° C., preferably at about 85° C. to about 160° C., most preferably at about 100° C. to about 140° C.; preferably for about 1 second to about 1 hour, more preferably about 1 second to about 30 minutes, most preferably about 1 second to about 15 minutes. Epimerization may be maximized while minimizing the total heating process effects when performed at a pH of about <6.0, preferably at a pH of about 3.8 to about 6.0, most preferably at a pH of about 5.0 to about 6.0; at a temperature of about 72° C. to about 200° C., preferably at about 85° C. to about 160° C., most preferably at about 100° C. to about 140° C.; preferably for greater than about 1 hour, more preferably greater than about 4 hours, most preferably, greater than about 6 hours.

It will be understood that, while the maximum temperature discussed in the following examples is 125° C., higher temperatures, e.g., up to approximately 200° C., may be used to further maximize the level of epimerization.

### Cocoa Ingredients

When the food product is a cocoa product, it may be in the form of a food or beverage containing partially defatted or fully defatted cocoa solids, chocolate liquor, and/or a cocoa extract. In this embodiment, the food product preferably is a dark chocolate bar, a dairy dessert, or a beverage. Also in this embodiment, the cocoa solids, chocolate liquor and/or cocoa extracts preferably are prepared from unfermented and/or underfermented cocoa beans.

When the cocoa product is an epimerized cocoa extract or an epimerized cocoa powder, preferably the molar ratio of catechin to epicatechin is greater than about 0.42 to 1, preferably the molar ratio of catechin to epicatechin is greater than about 0.54 to 1, and most preferably the molar ratio of catechin to epicatechin is greater than about 1 to 1.

Thermally processed cocoa ingredients are used in the high CP food products. When the products are a low moisture content product, they contain at least about 6 milligrams, preferably about 8, and more preferably about 10 milligrams of cocoa polyphenols per gram of the product, and the epicatechin to catechin ratio in the product is 1 to greater than 1. Preferably they contain at least about 10 milligrams, more preferably about 12, and most preferably about 14 milligrams of cocoa polyphenols per gram of the product, and the epicatechin to catechin ratio in the product is 1.0 to greater than 0.66. More preferably they contain at least about 12 milligrams, more preferably about 14, and most preferably about 16 milligrams of cocoa polyphenols per gram of the product, and the epicatechin to catechin ratio in the product is 1.0 to greater than 0.54. Even more preferably, they contain at least about 13 milligrams, more preferably about 15, and most preferably about 17 milligrams of cocoa polyphenols per gram of the product, and the epicatechin to catechin ratio in the product is 1.0 to greater than 0.42.

The high CP cocoa ingredients include a thermally-processed, partially defatted or fully defatted high CP cocoa powders which comprise (±)-catechin and (±)-epicatechin, and procyanidin oligomers thereof, which have a total CP content of at least about 25 milligrams, preferably about 12 to about 25 milligrams of cocoa polyphenols per gram of the defatted cocoa powder.

When the products are high moisture content foods such as a beverages (containing >50% moisture), they contain at least about 0.2, preferably 0.2 to 0.4, or more preferably 0.4 to 0.8. or most preferably 0.8 to 1.2 milligrams of total cocoa polyphenols per gram of the product.

As with the low moisture foods, the epicatechin to catechin content of the high moisture foods varies depending upon the cocoa polyphenol content of the product. Typically, products which contain about 0.2 to 0.4 milligrams have a ratio of 1 to greater than 1, the products which contain about 0.4 to about 0.8 milligrams have a ratio of 1 to 0.42, products which contain about 0.8 to about 1.2 milligrams have a ratio of about 1 to about 0.54, and the products which contain about 1 to greater than 1.2 milligrams to about 0.66 have a ratio of about 1 to about 0.66.

The ingredients also include thermally-processed high CP cocoa extracts, dry or liquid, which have a total CP content of at least about 200 milligrams, preferably about 250 to about 500, most preferably about 350 to about 500, per gram of the dry cocoa extract. The extracts also have altered profiles compared to cocoa extracts that have not been thermally-processed.

The ingredients also include thermally-processed chocolate liquor. The chocolate liquor contains at least about 10 milligrams of cocoa polyphenols per gram of the defatted cocoa liquor, preferably about 20 to about 50 milligrams, more preferably about 13 to about 17 milligrams.

While the specific examples disclosed were conducted on cocoa products, the methods for controlling epimerization disclosed and claimed herein may be used with any edible product containing epicatechin or catechin. Such products include but are not limited to fruit products, vegetable products, cereal products, bean products, nut products, spice products and botanical products, and the extracts thereof. The extracts are composed of flavanol monomers and proanthocyanidins, and preferably comprise catechin, epicatechin and procyanidins. Examples of epicatechin/catechin-containing fruit products include blueberry, cranberry, blackberry, raspberry, strawberry, bilberry fruit, black currant, cherry, grape, apple, apricot, kiwi, mango, peach, pear and plum. Examples of suitable vegetable products include Indian squash. Examples of suitable cereal products include sorghum and barley. Examples of suitable bean products include black-eyed peas, pinto beans, small red beans, and red kidney beans. Examples of suitable nut products include almonds, cashews, hazelnuts, pecans, walnuts, pistachios, and peanuts. Examples of suitable spice products include curries and cinnamon. Examples of suitable botanical products include Chinese hawthorn, Acacia catechin, Pterocarpus marsupium, Cassia Nomane, rhubarb, rhodiola, pine bark, willow bark and Uncaria tomentosa (cat's claw).

The following procedures were used for the preparation and testing of the products.

### Preparation of Samples

Normal Phase Chromatography-HPLC/MS Analysis (Adamson et al. method)

For Example 8, the published normal phase HPLC method of Adamson et al. (J. Agric. Food Chem., 1999, 47 pp. 4184-4186) was used. Conditions were as follows:

### a) Column:

### Phenomenex Lichrosphere Silica

Size: 25 cm×4.6 mm
Particle size: 5 micron

### Pore Size: 100 Angstrom

### b) Mobile Phase:

### A. Methylene Chloride

### B. Methanol

### C. Water:Acetic Acid (1:1)

### Gradient Conditions:

### Initial: 82% A/14% B/4% C

Time=30 mins. 67.6% A/28.4% B/4% C
Time=50 mins. 53.2% A/42.8% B/4% C
Time=51 mins. 10% A/86% B/4% C
Time=56 mins. 82% A/14% B/4% C
Re-equilibration-7 minutes
c) Flow Rate: 1.0 ml/min

### d) Column Temperature: 37° C.

e) Injection Volume: 5.0 microliters

### f) Detection:Fluorescence: Excitation Wavelength 276 nm:Emission Wavelength-316 nm

### Normal Phase Chromatography: Diol Method

For the other examples, the normal phase chromatography employed was a halogen free method generally referred to as the DIOL method. The method is disclosed in “High-Performance Liquid Chromatography Separation and Purification of Cacao (Theobroma cacao L.) Procyanidins According To Degree of Polymerization Using a Diol Stationary Phase” by M. A. Kelm, et al., (J. Agr. & Food Chem. (2006) 54(5), 1571-6). Conditions were as follows:

Analyitical Normal-Phase HPLC method

Name: CPDIOL-3.M

Column: Intersil Diol 250×4.6 mm

Mobile Phase A 98:2 acetonitrile:acetic acid

Mobile Phase B 95:3:2 methanol:H₂O:acetic acid

Flow Rate:

| | Time (min) | % B |
|---|---|---|
| Gradient | 0 | 0 |
| | 35 | 40 |
| | 45 | 40 |
| | 46 | 100 |
| | 50 | 100 |

The column used was a 250×4.6-mm, i.d., 5 μm Develosil diol (Phenomenex, Torrance, Calif.). The binary mobile phase consisted of (A) CH₃CN:HOAc, (98:2, v/v) and (B) CH₃OH:H₂O:HOAc (95:3:2). Separations were effected by a linear gradient at 30° C. with a 1.0 mL/min flow rate as follows: 0-35 min, 0-40% B; 35-45 min, 40% B isocratic; 45-46 min, 40-0% B, 4 min hold at 0% B. Eluent was monitored by fluorescence detection with excitation at 276 nm and emission at 316 nm.

Reversed Phase High Pressure Liquid Chromatography-C18 Method

An Agilent 1100 LC instrument coupled with photodiode array, fluorescence detector, and quadrapole MS was used for the separation and detection of the monomers and procyanidins, as well as the determination of epicatechin to catechin ratios in the unfermented cocoa beans, cocoa extracts, uncooked and cooked cocoa powder, and the cocoa drinks. A Hypersil ODS (C18, 100×4.6 mm, 5 μm) column was employed. The mobile phase consisted of A (1% acetic acid in water) and B (0.1% acetic acid in methanol) using linear gradients of 10-25% B (v/v) for 20 min followed by an increase to 100% B for 10 min and up to 100% B for 10 min. The flow rate was set to 1.0 mL/min. The column over temperature was set at 20° C. The UV detector was set at 280 nm to record peak intensity, and UV spectra were recorded from 200-600 nm. The ionization technique was electrospray (ESI) and the mass spectrum data was all acquired in negative ion mode. For the quantitative work, the calibration curves were established using this chromatography and FLD detection. Eluent was monitored by fluorescence detection with excitation at 276 nm and emission at 316 nm.

### Example 1

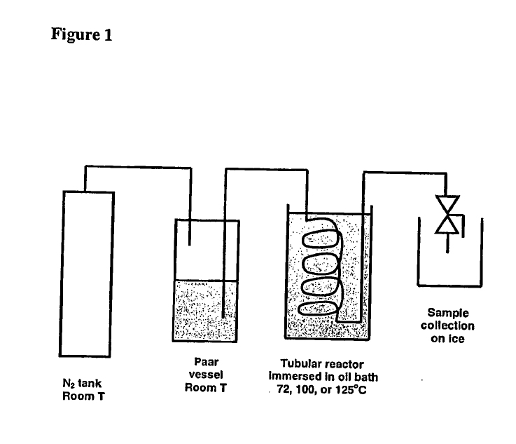

A 1 mg/ml solution of (−)-epicatechin (purchased from Sigma Aldrich) in buffered solution (sodium phosphate for pHs 6 and 7, sodium citrate for pH 4) was placed in a tubular reactor, with the epimerization occurring under a controlled atmosphere. FIG. 1 shows a schematic diagram of the reactor used.

Epimerizations were performed under a nitrogen atmosphere to avoid the loss of (−)-epicatechin by oxidation. Nitrogen gas was used to create pressure inside the feed vessel, pushing the solution into a tubular reactor immersed in an oil bath heated at the desired temperature. Fast heat transfer, provided by the thin design of the tubular reactor, guaranteed almost immediate heating of the (−)-epicatechin to the desired temperature. Aliquot samples of 5 ml each were collected over the course of the reaction, placed on ice, and quenched with 10 N HCl to prevent oxidation during compositional analysis.

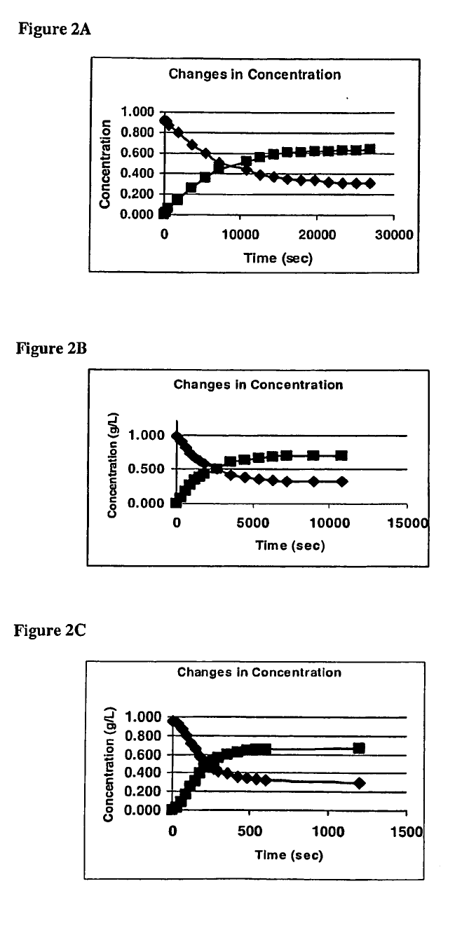

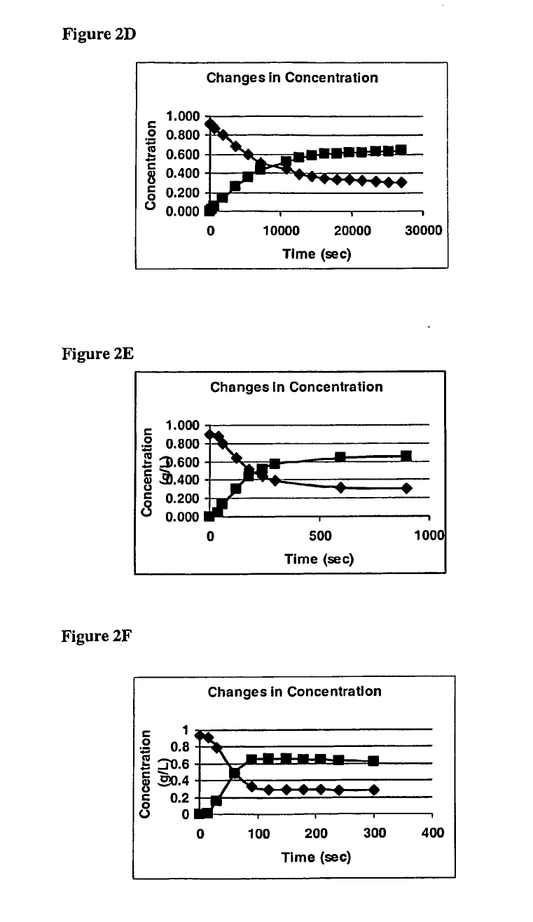

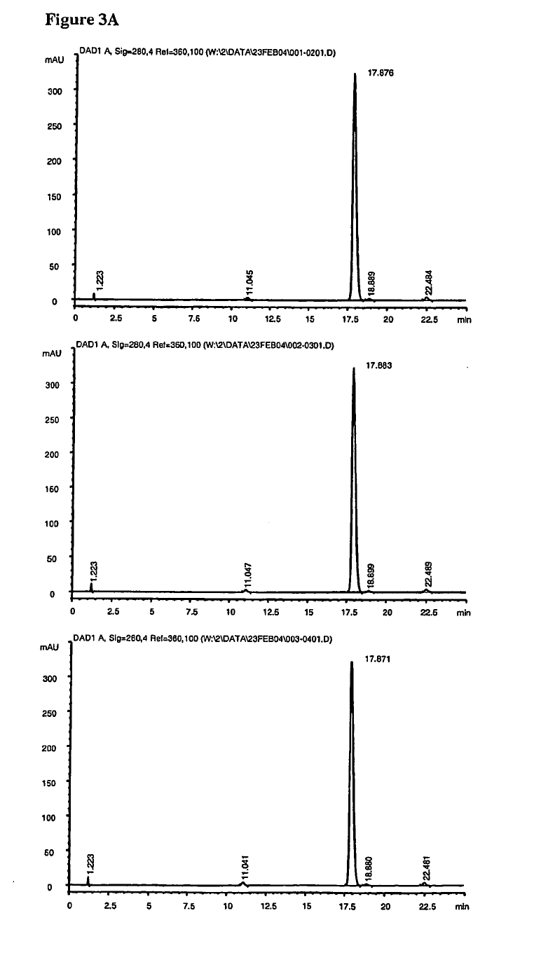

The composition of the collected (−)-epicatechin/(−)-catechin mixed samples was determined by HPLC analysis using (−)-epicatechin and (+)-catechin standards. FIGS. 2A through 2F show the change in concentration of (−)-epicatechin and (−)-catechin over the course of the epimerization under specific temperature and pH conditions. In all figures, concentration of (−)-epicatechin is represented by dark diamonds, while the concentration of (−)-catechin is represented by dark squares. As shown, under all reaction conditions, equilibrium represented a mixture of about one-third (−)-epicatechin and about two-thirds (−)-catechin; that is, about 70% of the (−)-epicatechin was lost due to epimerization. At equilibrium, the molar ratio of (−)-epicatechin to (−)-catechin is approximately 1:2.

The rate of epimerization differed significantly as a function of pH and temperature. Reactions were conducted at three pH levels: 4, 6 and 7; and at three temperatures: 72, 100 and 125° C. As shown in FIGS. 2A through 2F, the rate at which equilibrium was reached was highest at pH 7 (neutral) and at the highest temperature (125° C.). The table below shows the time at which equilibrium was reached for all conditions:

| Temperature (° C.) | pH | Time for equilibrium | |
|---|---|---|---|
| 72 | 4 | >15 | days |
| 72 | 6 | 2.5 | days |
| 72 | 7 | 6 | hours |
| 100 | 4 | 4 | days |
| 100 | 6 | 2 | hours |
| 100 | 7 | 10 | minutes |
| 125 | 4 | 6.5 | hours |
| 125 | 6 | 10 | minutes |
| 125 | 7 | 1.5 | minutes |

As shown in the table, the epimerization of (−)-epicatechin to (−)-catechin was strongly influenced by pH and temperature. For example, the loss of (−)-epicatechin reached its maximum (70%) within only 1.5 minutes at a neutral pH when subjected to retorting temperature.

Reaction rate increased by an order of magnitude when the temperature was raised to 100° C. at pH 7.

Data for epimerization at the reaction parameters of pH=4 and temperature=37° C. were excluded, as the rate of epimerization of (−)-epicatechin to (−)-catechin was decreased to such an extent as to be too long to visualize a change in the concentration of (−)-epicatechin.

### Example 2

Epimerization under Physiological pH and Temperature (37° C., pH 7.4): 50 mg of (−)-epicatechin (Aldrich) was dissolved in 50 ml pH 7.4 sodium phosphate buffer (Fluka, diluted ten times). Methanol (1 ml) was used to aid in dissolution of the epicatechin. 10 ml headspace vials (Supelco) containing aliquots of the epicatechin solution were hermetically sealed and purged with nitrogen gas, in order to prevent oxidation, via a needle inserted through the septum to provide nitrogen flow, and a second needle to promote venting. The vials were then placed in heater blocks set to 37° C. and mounted on an orbital shaker. The reaction was allowed to proceed over time, with samples collected at 15, 30, 60, 90, 120, 180 minutes and 48 hours. Samples were prepared and analyzed by HPLC, as follows:

Sample Preparation: Aqueous samples were filtered through 0.45 μm PTFE syringe filters into 1.8 ml autosample vials and sealed. Samples either were analyzed immediately or were stored frozen until analyzed, to avoid loss of (−)-epicatechin through oxidation.

HPLC conditions: HPLC analyses were performed on a 200×4.6 mm 5 μm Hypersil ODS column at 35° C. Separations were effected using a gradient elution with a binary mobile phase of (A) water:acetic acid 99:1 (v/v) and (B) water:methanol 88:12 (v/v), according to the gradient profile in the table below. After each run, the system was recalibrated for 7 minutes prior to the next run.

| Time (min) | % A | % B |
|---|---|---|
| 0 | 88 | 12 |
| 1 | 88 | 12 |
| 23 | 65 | 35 |
| 25 | 50 | 50 |

Detection and quantification of individual epimers were carried out at λ=280±4 nm and a reference of λ=360±100 nm. External standard least square calibration curves were generated for catechin and epicatechin by injecting 3 μl of standard solutions containing both analytes at 0.02, 0.1, and 1.0 mg/ml, respectively, and plotting area versus concentration.

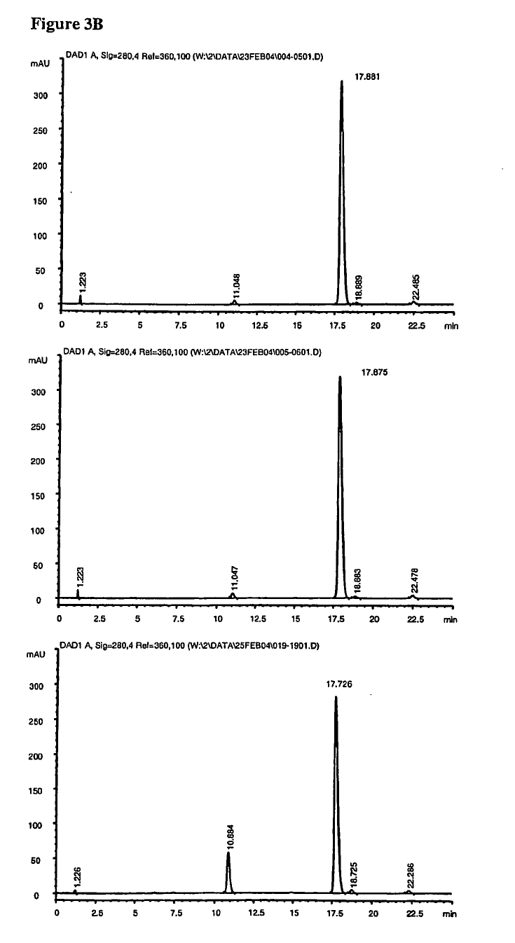

FIGS. 3A and 3B depict the HPLC chromatograms showing time epimerization profiles at pH 7.4, 37° C. FIG. 3A shows epimerization profiles at 15 (top), 30 (middle) and 60 (bottom) minutes. FIG. 3B shows epimerization profiles at 120 minutes (top), 180 minutes (middle) and 48 hours (bottom). As shown in FIG. 3B, even after a reaction time of 48 hours, epimerization of (−)-epicatechin to (−)-catechin had not reached equilibrium.

### Example 3

Low Water Activity, 72° C. and 125° C., pH 7 (non-kinetic): 300 mg of (−)-epicatechin (Aldrich) was dissolved in mixture of 30 ml pH 7 sodium phosphate buffer and 270 ml ethylene glycol to obtain final water activity of 0.2. Methanol (3 ml) was added to aid in dissolution.

A stirred Paar reactor vessel (Model no. 4841) containing the epicatechin solution was purged with nitrogen gas for approximately 15 minutes and then placed in its mantel heater, set to the target temperature (72° C. or 125° C.). The epimerization reaction was allowed to proceed over time while the temperature progressively rose to the target value. (We note that the temperature of the mantel heater could not be set in advance. The mantel heater was regulated by the internal sample temperature, and the reactor's thick steel walls made heat transfer inefficient and slow. In one case, a target temperature of 125° C. was overshot to 158° C.) Samples were collected by opening the outlet valve at timed intervals, placed over ice for rapid cooling, acidified to pH 3.8 and submitted to HPLC analysis using the sample preparation and analysis protocol set forth above.

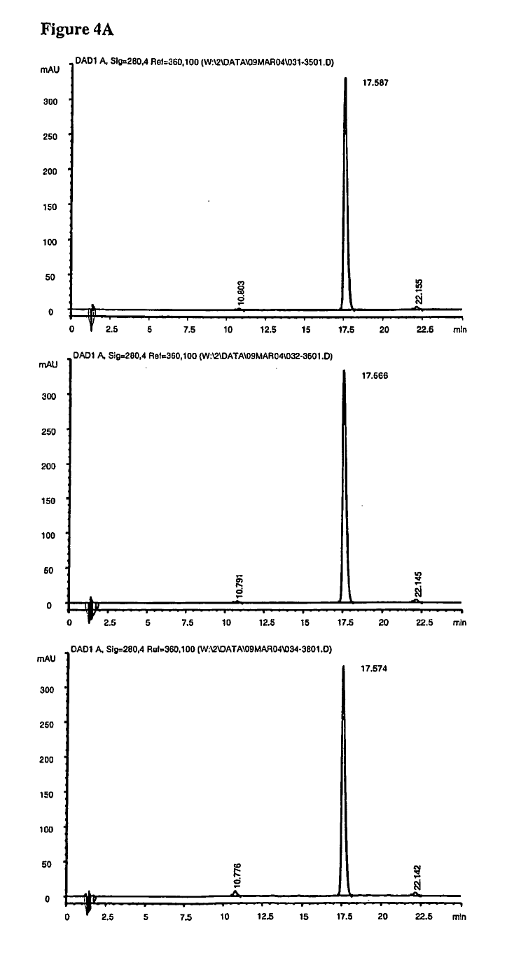

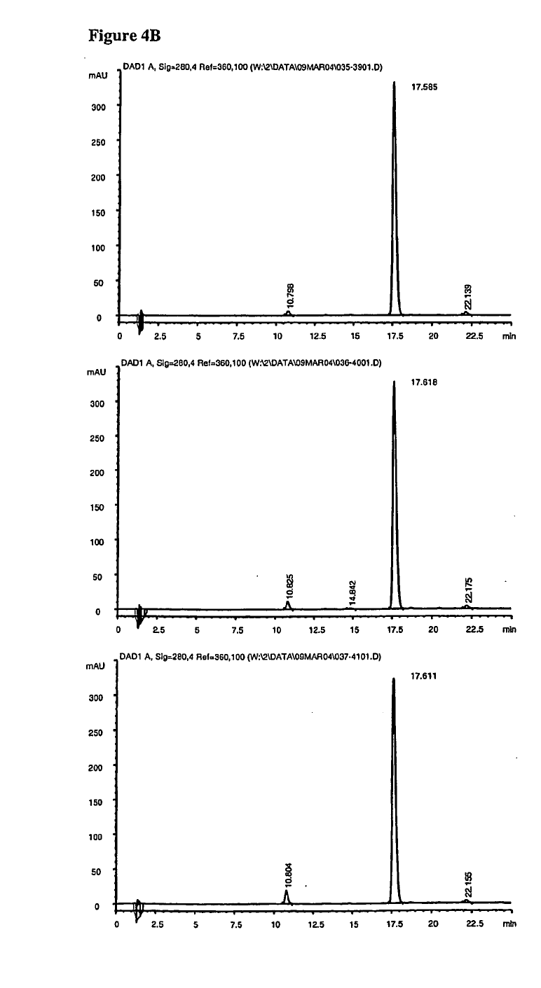

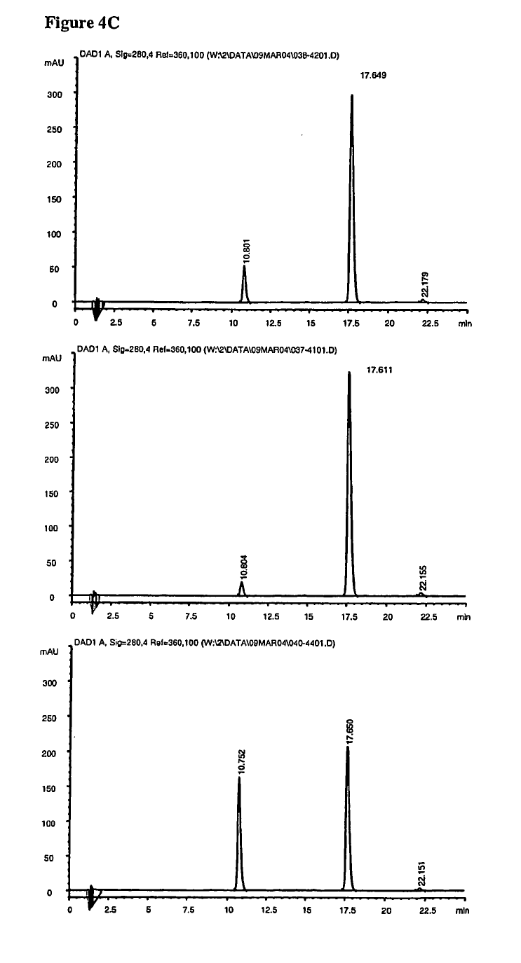

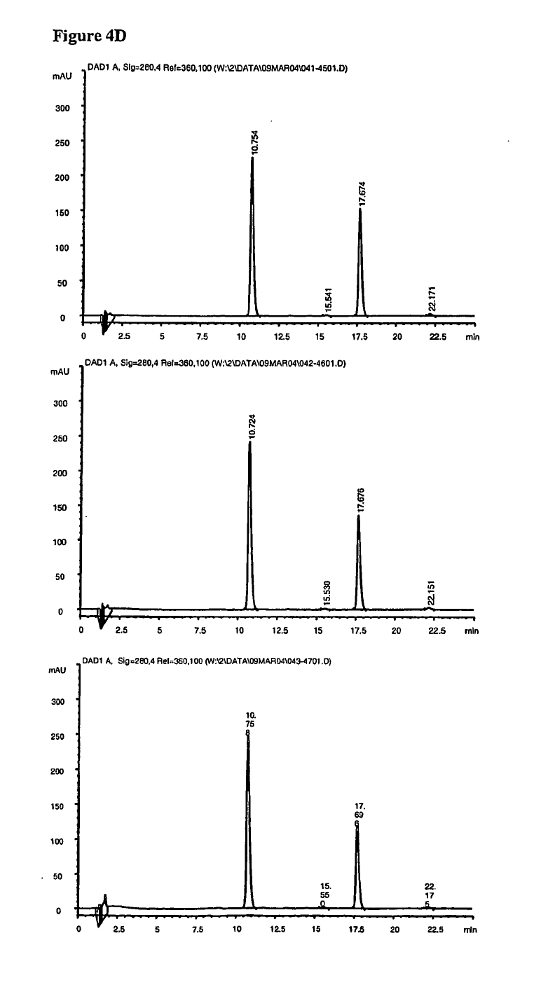

FIGS. 4A-D show that epimerization of (−)-epicatechin to (−)-catechin in a low water activity environment is significantly affected by reaction time and temperature. FIGS. 4A-D have the common reaction parameters of low water activity (0.2), pH 7.0. In FIG. 4A, the other reaction parameters are 30 seconds at 23° C. (top), 1 minute at 37° C. (middle), 2 minutes at 62° C. (bottom). In FIG. 4B, the other reaction parameters are 2.5 minutes at 77° C. (top), 3 minutes at 85° C. (middle), 3.5 minutes at 93° C. (bottom). In FIG. 4C, the other reaction parameters are 4 minutes at 100° C. (top), 4.5 minutes at 108° C. (middle), 5 minutes at 116° C. (bottom). In FIG. 4D, the other reaction parameters are 6 minutes at 126° C. (top), 7 minutes at 135° C. (middle), 8 minutes at 140° C. (bottom).

Comparing FIGS. 4A, B with FIGS. 4C, D, it is evident that epimerization is substantially more advanced at higher temperatures and longer reaction times (FIGS. 4C, D).

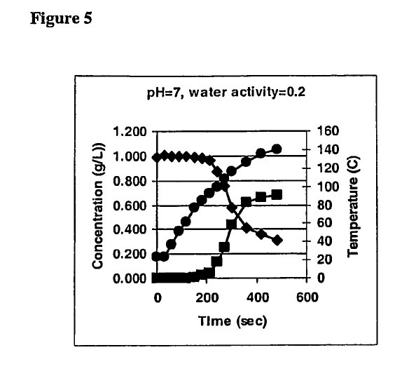

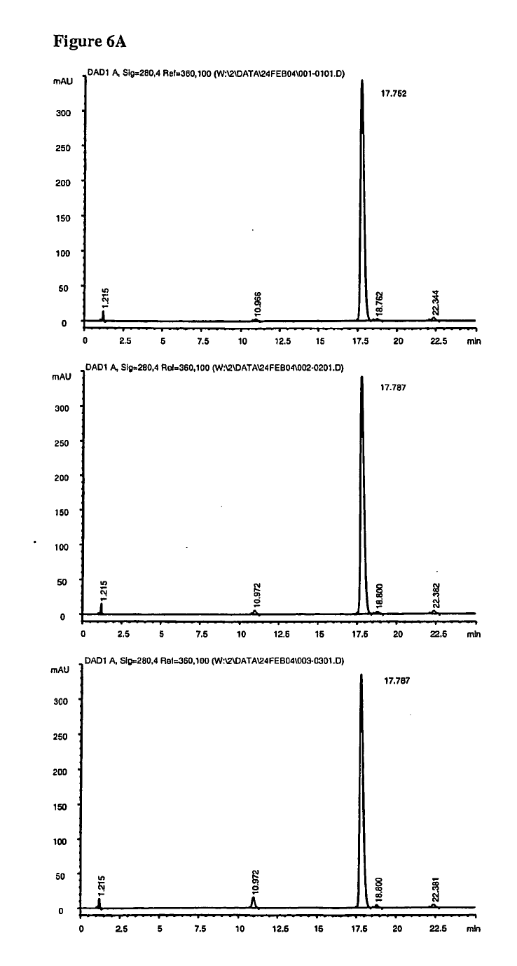

FIG. 5 shows that epimerization may be carried out in a low water activity environment. Similarly to the aqueous solutions, the concentrations of (−)-catechin and (−)-epicatechin are driven towards the equilibrium point at pH 7, under increasing temperature from ambient to 140° C. We note that FIG. 5 does not represent a kinetic experiment, from which reaction rate can be calculated, but rather confirms that the epimerization can be maximized to equilibrium, even in a medium of water activity as low as 0.2.

### Example 4

Food Processing Conditions; 72° C., 100° C., 125° C., pH 4, 6, 7: Solutions of (−)-epicatechin (1 mg/ml, Aldrich) were prepared using phosphate (pH 6 and pH 7) and citrate (pH 4) buffers. Methanol (2 ml) was added to aid in dissolution of (−)-epicatechin in the buffer. About 100 ml of a given epicatechin solution was placed in a Paar reactor vessel (Model No. 4841), which was then purged with nitrogen gas for approximately 15 minutes. The Paar vessel was connected with a coiled length of ⅛-inch stainless steel tubing. After nitrogen purging, a flow of the epicatechin solution was allowed through the stainless tubing, which has capacity for approximately 100 ml of liquid. The filled coiled tubing was immersed in a large heated oil bath at the target temperature (72° C., 100° C., 125° C.) and the reaction was allowed to proceed. The Paar vessel was kept under pressure in order to push aliquots out of the coiled tubing reactor at pre-selected sampling times. Samples were collected at timed intervals, placed over ice for rapid cooling, acidified to pH 3.8, and submitted to HPLC analysis, using the sample preparation and analysis protocol set forth above.

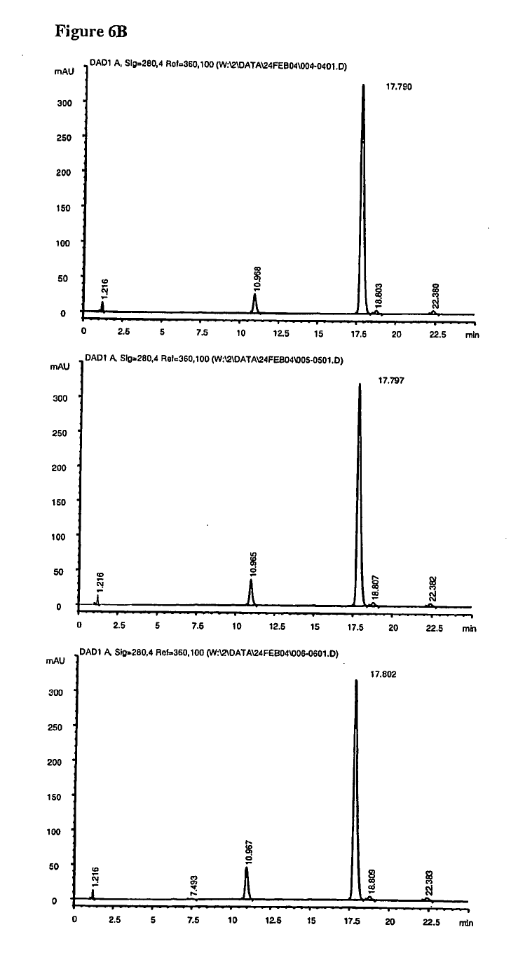

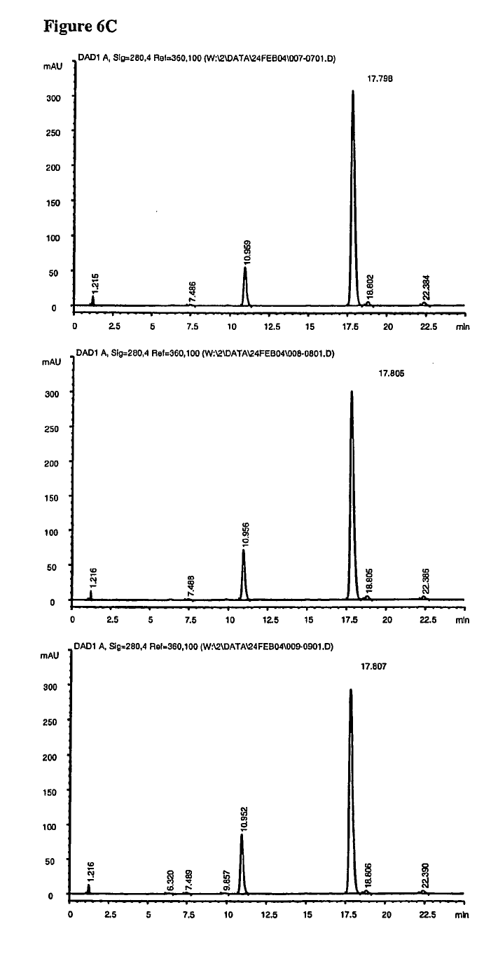

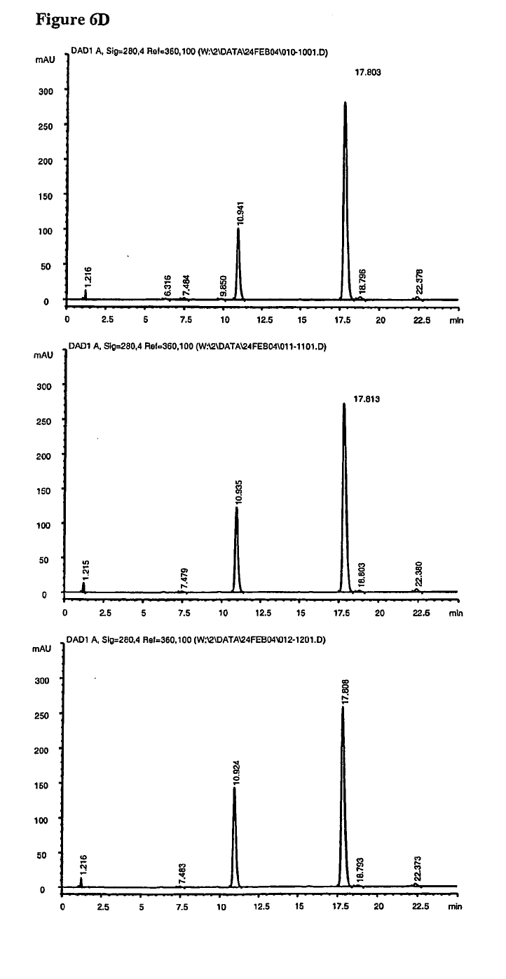

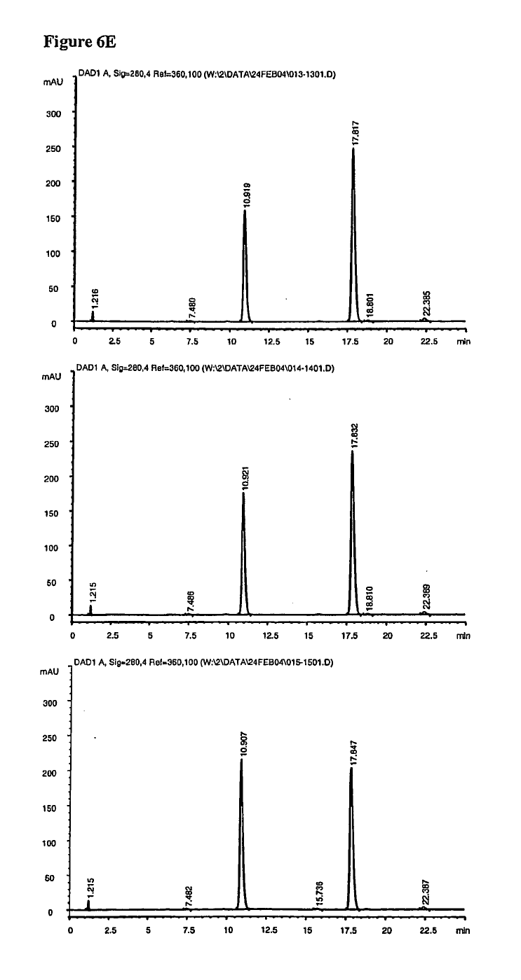

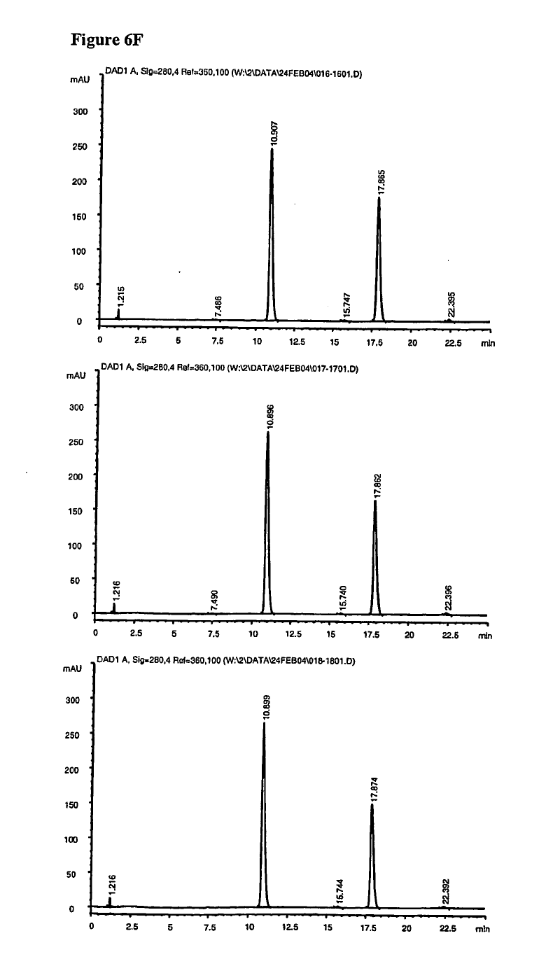

FIGS. 6A-F show the time profiles for the epimerization of (−)-epicatechin to (−)-catechin at pH 7.0, 72° C., at various reaction times. In FIG. 6A, reaction times are 0 (top), 5 (middle) and 10 (bottom) minutes. In FIG. 6B, reaction times are 15 (top), 20 (middle) and 25 (bottom) minutes. In FIG. 6C, reaction times are 30 (top), 40 (middle) and 50 (bottom) minutes.

In FIG. 6D, reaction times are 60 (top), 75 (middle) and 90 (bottom) minutes. In FIG. 6E, reaction times are 105 (top), 120 (middle) and 180 (bottom) minutes. In FIG. 6F, reaction times are 240 (top), 300 (middle) and 360 (bottom) minutes.

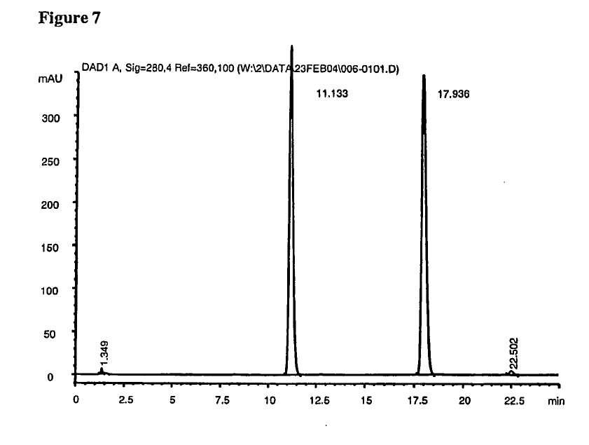

As expected, FIGS. 6A-F confirm that epimerization of (−)-epicatechin to (−)-catechin at pH 7.0, 72° C. did not reach equilibrium until after 300 minutes. (Compare FIG. 7, showing the catechin-epicatechin standard).

### Example 5

Cocoa polyphenol extract, pH 3.8: 200 mg Cocoa polyphenol (CP) extract derived from unprocessed cocoa were dissolved in 200 ml water. One ml methanol was used to aid the dissolution. The final pH of this solution was 3.8.

A stirred Paar reactor vessel (Model No. 4841) containing 100 ml of the CP extract solution was purged with nitrogen gas for 20 minutes and then placed in its mantle heater set to the target temperature (100° C.). The temperature rose over time to 102° C. The reaction was allowed to take place for 60 minutes once the temperature 102° C. was reached. At the end of the 60 minutes of reaction at 102° C., samples were collected by opening an outlet valve into a pre-chilled 150 ml Erlenmeyer flask placed in an ice bath. Once cooled, an aliquot of the reacted sample, as well as the stock (unreacted) solution, were submitted to HPLC analysis, using the sample preparation and analysis protocol set forth above.

### Example 6

Cocoa polyphenol extract, pH 7: 200 mg CP extract derived from unprocessed cocoa were thoroughly dispersed in approximately 10 ml water. One ml methanol was used to aid the dispersion. The volume was completed to 200 ml by adding a sodium phosphate buffer. The final pH of this solution was 7.0. An aliquot of the starting (unreacted) solution was acidified with hydrochloric acid to pH 2.5.

A stirred Paar reactor vessel (Model No. 4841) containing 100 ml of the CP extract solution was purged with nitrogen gas for 20 minutes and then placed in its mantle heater set to the target temperature (100° C.). The temperature rose over time to 102° C. The reaction was allowed to take place for 60 minutes once the temperature 102° C. was reached. At the end of the 60 minutes of reaction at 102° C., samples were collected by opening an outlet valve into a pre-chilled 150 ml Erlenmeyer flask placed in an ice bath. Once cooled, an aliquot of the reacted sample was acidified with hydrochloric acid to pH 2.5. Both the reacted and unreacted acidified solutions were then submitted to HPLC analysis, using the sample preparation and analysis protocol set forth above.

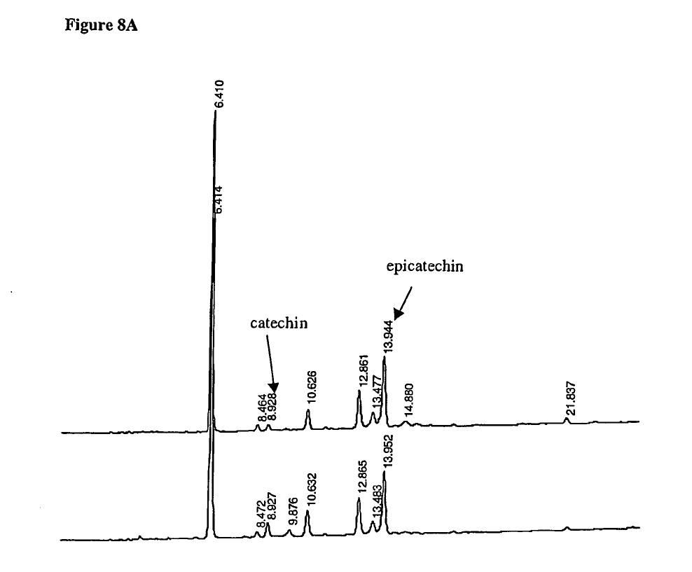

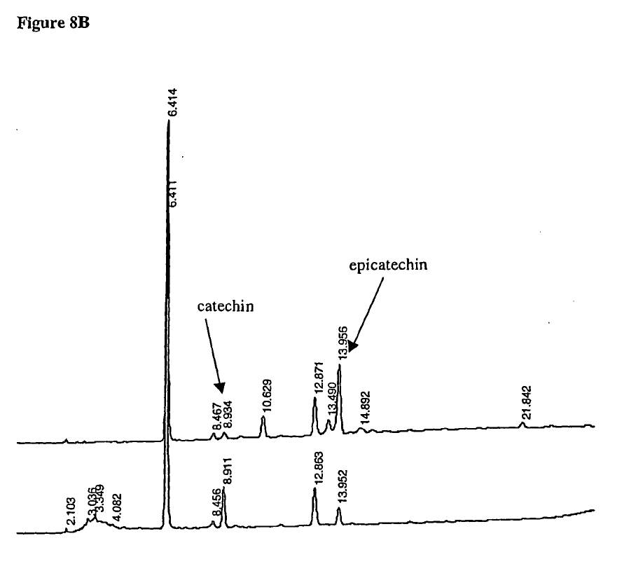

FIGS. 8A and 8B show the epimerization of (−)-epicatechin into (−)-catechin in the CP extract. A comparison between the pH 3.8 (FIG. 8A) and the pH 7.0 (FIG. 8B) confirms that the epimerization in the extract is accelerated at the higher pH, and delayed at the lower pH, which is in agreement with the results in Examples 1-4, where the epimerization was carried out on a pure solution of (−)-epicatechin. In each of FIGS. 8A and 8B, the top chromatogram depicts the unreacted CP extract at the given pH, while the bottom chromatogram depicts the reacted CP extract.

### Example 7

Epimerization of (+)-Catechin to (+)-Epicatechin. A 1 mg/ml solution of (+)-catechin was prepared in buffered solution, pH 7.5, measured at 21° C. as follows: 5 mg (+)-catechin (purchased from Sigma Aldrich, 98% minimum purity) was dispersed in 200 mL ethanol (190 proof) and 4.8 mL phosphate buffered saline solution were added to a final concentration of 1 mg/ml (+)-catechin. The phosphate buffered saline solution was prepared by dissolving one phosphate buffered saline tablet (purchased from Sigma Aldrich) in 200 ml milli-Q HPLC-grade water to yield nominal concentrations of 137 mM sodium chloride, 2.7 mM potassium chloride and 10 mM phosphate buffer. The (+)-catechin solution was placed in a hermetically sealed vial capped with a septum. The vial was purged with nitrogen gas via an injection needle inserted through the septum into the liquid, and a purge needle inserted through the septum into the headspace and sealed following purging. The vial was incubated overnight on a block heater set to 80° C. and mounted on an orbital shaker to promote agitation followed by HPLC determination of epimer concentrations, as described above.

The (+)-catechin final concentration was 0.64 mg/mL, and the (+)-epicatechin final concentration was 0.36 mg/mL. These results represent an equilibrium molar ratio of about 2:1, (+)-catechin:(+)-epicatechin, similar to the examples depicting the kinetics for epimerization of (−)-epicatechin to (−)-catechin, viz., equilibrium for a given set of temperature and pH parameters is essentially the same for either epimerization, and the equilibrium mixture resulting from the epimerization of (+)-catechin is about 70% (+)-catechin and about 30% (+)-epicatechin, with a molar ratio of (+)-epicatechin to (+)-catechin of about 1:2. Indeed, we have observed that the epimerization of (+)-catechin to (+)-epicatechin favors the same equilibrium point as the epimerization of (−)-epicatechin to (−)-catechin, that is, the molar ratio of 1:2 (+)-epicatechin:(+)-catechin.

### Example 8

Preparation of High CP Cocoa Solids from Cocoa Beans. Commercially available cocoa beans having an initial moisture content of about 7 to 8% by weight were pre-cleaned in a scalperator. The pre-cleaned beans from the scalperator were further cleaned in an air fluidized bed density separator. The cleaned cocoa beans were then passed through an infra-red heating apparatus at a rate of about 1,701 kilograms per hour. The depth of beans in the vibrating bed of the apparatus was about 2-3 beans deep. The surface temperature of the apparatus was set at about 165° C., thereby producing an internal bean temperature (MT) of about 135° C. in a time ranging from 1 to 1.5 minutes. This treatment caused the shells to dry rapidly and separate from the cocoa nibs. The broken pieces separated by the vibrating screen prior to the apparatus were re-introduced into the product stream prior to the winnowing step. The resulting beans after micronizing should have a moisture content of about 3.9% by weight. The beans emerged at an IBT of about 135° C. and were immediately cooled to a temperature of about 90° C. in about three minutes to minimize additional moisture loss. The beans were then winnowed to crack the beans, to loosen the shells, and to separate the lighter shells from the nibs while at the same time minimizing the amount of nib lost with the shell reject stream. The resulting cocoa nibs were pressed using two screw presses to extract the butter from the cocoa solids.

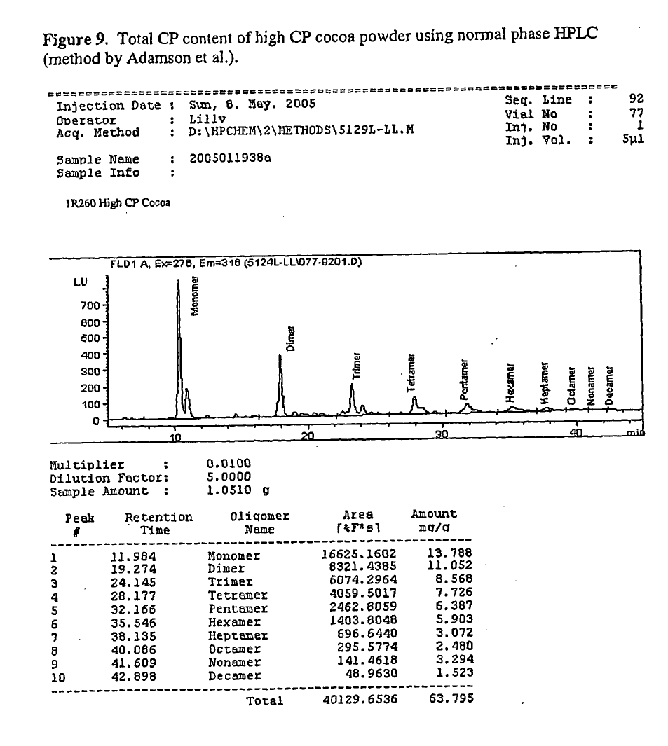

A sample of cocoa solids, produced according to the above-described process from unfermented cocoa beans (fermentation factor 233), when analyzed according to the above-referenced method, typically will have a total cocoa procyanidin content of about 50 to about 75, preferably about 60 to about 75, or more preferably about 75 to about 80 milligrams total cocoa procyanidins per gram of defatted cocoa powder. FIG. 9 shows the normal phase HPLC trace of the high CP cocoa powder.

### Example 9

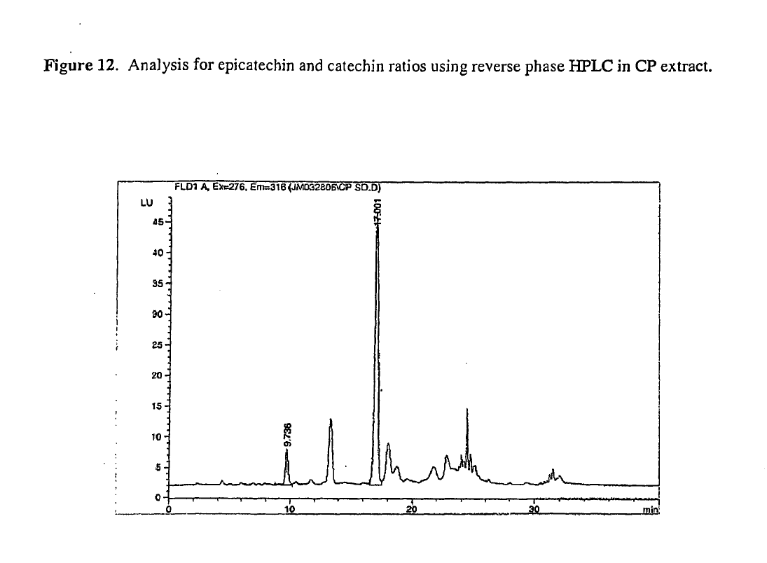

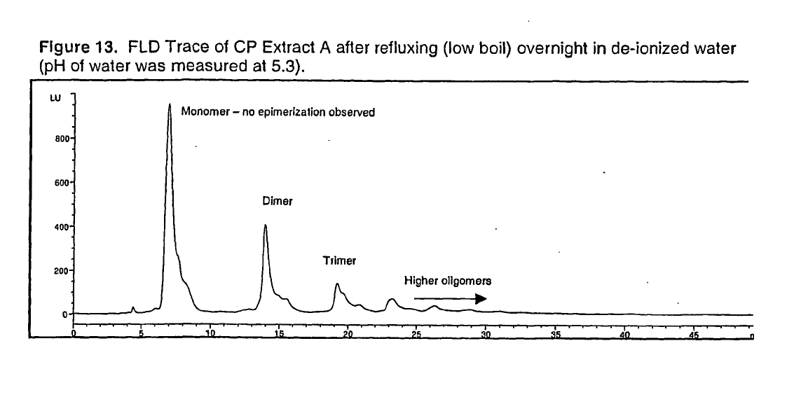

Preparation of Cocoa Extracts. The cocoa solids from Example 8 were contacted at room temperature for from 0.5 to 2.5 hours with an aqueous organic solvent. For Cocoa Extract A the solvent was about 75% ethanol/25% water (v/v). For Cocoa Extract B the solvent was about 80% acetone/20% water (v/v). The micella was separated from the cocoa residue and concentrated by evaporation. The concentrated extract was then spray dried. The HPLC/FLD profiles of the cocoa extracts are shown. FIG. 12 shows the trace prior to heating. FIG. 13 shows the trace for the ethanol extract after refluxing overnight in deionized water.

### Example 10

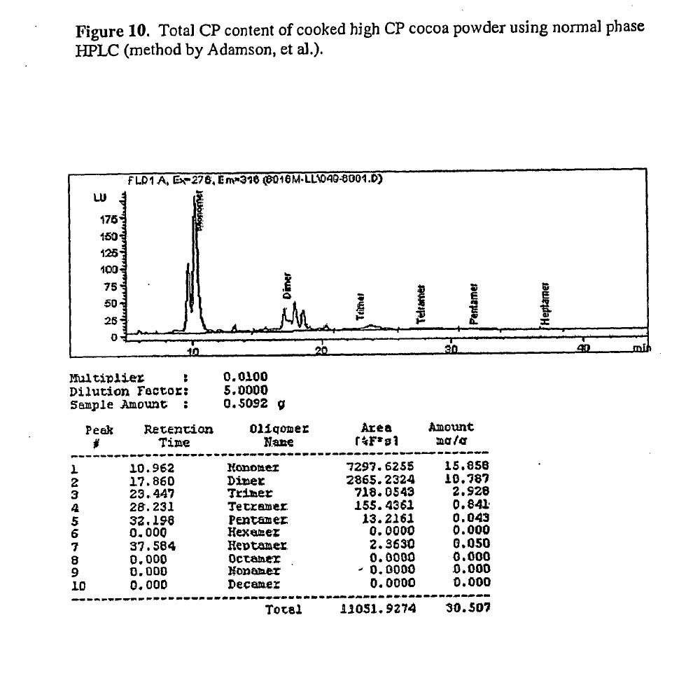

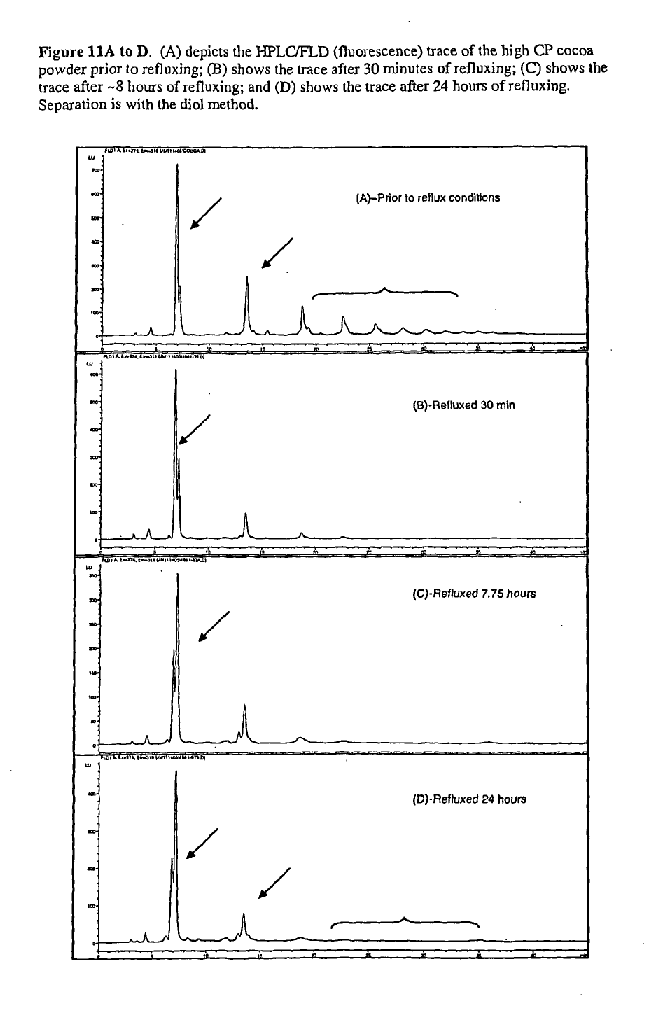

LCMS investigation of procyanidin chemistry in high CP partially defatted cocoa powder. A high CP cocoa powder (50 g) was suspended in 500 mL of de-ionized water (pH 5.3) in a 1 L round bottom flask equipped with a water cooled condensor. A heat mantel was used as the heat source and the mixture was refluxed. Samples were taken at 30 min, 7.75 hours, and 24 hours. The normal phase HPLC/FLD trace of the original high CP cocoa powder is shown in FIG. 9. Separation was with the diol method. FIG. 10 shows the normal phase HPLC trace for the cooked high CP cocoa powder (Method of Adamson et al.). FIGS. 11A to D show the traces prior to cooking and after cooking for 30 min, 7.75 hours, and 24 hours.

The total CP content of the high CP cocoa powder prior to any processing was ˜57 mg/g or ˜6%. The CP content was measured using the method of Adamson et al. The polyphenols measured included the monomer through decamer. Once cooked the total CP content was reduced to 30 mg/g. Monomer content determined from this data shows that were 13.79 mg/g of monomers present in the uncooked high CP cocoa powder (1.4% monomer by mass) and that the monomer amount was unchanged after cooking, with the amount being 15.8 mg/g (1.6% by mass).

Quantitation using reverse phase (RP) HPLC (C18) was performed as well. See FIG. 15. Calibration curves were established with authentic standards. The monomer amounts for the uncooked high CP cocoa powder were fairly consistent with the amounts determined under normal-phase conditions. As a control, the monomeric fraction was isolated from the cooked high CP cocoa powder using a preparative diol column. Reverse phase analysis of the purified fraction isolated from the cooked high CP cocoa powder showed a clean trace of epicatechin and catechin. The results are shown in Table 1.

TABLE 1

| | | | | Monomers |
|---|---|---|---|---|
| | | | | Based on |
| | | | Total | Sample |
| | Catechin | Epicatechin | Monomers | Mass |
| | (mg/mL) | (mg/mL) | (mg/mL) | (%) |
| High CP | 1.70 × 10⁻³ | 1.01 × 10⁻² | 1.18 × 10⁻² | 1.14% |
| cocoa powder | 3.13 × 10⁻³ | 1.21 × 10⁻² | 1.52 × 10⁻² | 1.52% |
| Cooked high CP | 9.86 × 10⁻³ | 3.12 × 10⁻³ | 1.30 × 10⁻² | 1.27% |
| cocoa powder | 8.18 × 10⁻³ | 4.80 × 10⁻³ | 1.30 × 10⁻² | 1.27% |
| Monomeric | 0.323 | 0.176 | 0.500 | 50% |
| fraction | | | | |
| isolated from | | | | |
| cooked high CP | | | | |
| cocoa powder | | | | |

### Example 11

Investigation of ratio of epicatechin to catechin. The ratio of epicatechin to catechin was measured using C18 HPLC methodology. The various cocoa products tested included unfermented cocoa beans, two high CP cocoa extracts, uncooked and cooked high CP cocoa powder, and Cocoa Drink A. Cocoa Extract A was prepared by extracting unfermented cocoa beans with aqueous ethanol (25% water/75% ethanol, v/v). Cocoa Extract B was prepared by extracting unfermented cocoa beans with aqueous acetone (20% water/80% aceteone, v/v). The ratios are shown in Table 2.

TABLE 2

| | Epicatechin:Catechin |
|---|---|
| Cocoa Product | Based on 100 |
| Unfermented cocoa beans | 95:5 |
| Cocoa Extract A | 96:4 |
| Cocoa Extract B | 90:10 |
| Uncooked high CP cocoa powder | 79:21 |
| Cooked high CP cocoa powder | 33:67 |

In the products that have not been thermally processed, e.g., the cocoa extracts, the epicatechin content is greater than the catechin content, which is consistent with what is observed in unfermented cocoa beans. For the high CP cocoa powder, the epicatechin to catechin ratio is 79:21. For the highly processed sample, i.e., the cooked high CP cocoa powder, a ratio of ˜35:65 epicatechin to catechin is reached. This ˜35:65 epicatechin to catechin ratio is the thermodynamic equilibrium of these two diastereomers for the epimerization reaction (catechin is naturally the more stable form). Thus, the degree of processing provides some insight into the degree of conversion of epicatechin to catechin. With minor or no processing, the ratio of epicatechin to catechin is ˜95:5. With more processing, particularly high temperature processing, the ratio shifts to ˜80:20 as with the uncooked high CP cocoa powder. With high processing, the ratio reaches the equilibrium point.

### Example 12

Investigation of Chiral Content. In order to determine the ratio of stereoisomers, chiral chromatography was performed. The ratio of (+/−)-catechin was obtained under one set of chromatographic conditions and that of (+/−)-epicatechin was obtained under a different set of chromatographic conditions.

The epicatechin and catechin observed in the various cocoa samples were further analyzed for stereochemical make up. The chiral content is provided in Table 3.

TABLE 3

| Cocoa product | (−)/(+)-Epicatechin | (+)/(−)-Catechin |
|---|---|---|
| Unfermented cocoa beans | 100:0 | 90:10 |
| High CP cocoa extract A | 100:0 | 39:61 |
| High CP cocoa extract B | 100:0 | 34:66 |
| Uncooked high CP cocoa | 100:0 | 13:87 |
| powder | | |
| Cooked high CP cocoa powder | 95:5 | 4:96 |

Catechin is a minor component in the cocoa bean and the naturally occurring ratio of (+)-catechin to (−)-catechin is 90:10. For catechin, the predominant form in the bean is (+)-catechin. In the two cocoa extracts the ratio changes to about 40:60 (+/−)-catechin which differs from the cocoa bean data—there is an increase in the presence of (−)-stereoisomer. Presumably, the source of the (−)-catechin is the conversion of (−)-epicatechin to (−)-catechin since the conversion is stereospecific. Further processing enhances the conversion until the predominant form is (−)-catechin. Processing enhances the (−)-catechin content until it becomes the predominant stereoisomer in the highly processed cocoa samples such as Cocoa Drink A. This is consistent with the expected conversion reaction since (−)-catechin is generated by the conversion of (−)-epicatechin under the heat processing conditions. (−)-Epicatechin is the only isomer observed in minorly processed materials. The stereoisomer, (+)-epicatechin, is observed in very small amounts in the highly processed cocoa samples. This is consistent with the fact that the (+)-epicatechin is expected to be generated from (+)-catechin and that (+)-epicatechin is the less stable stereoisomer. In order to determine the ratio of stereoisomers, chiral chromatography was performed. The ratio of (+/−)-catechin was obtained under one set of chromatographic conditions and that of (+/−)-epicatechin was obtained under a different set of chromatographic conditions. The results show that all four stereoisomers exist in varying amounts in the processed materials, i.e., the cooked high CP cocoa powder.

While the invention has been described with respect to certain specific embodiments, it will be appreciated that many modifications and changes may be made by those skilled in the art without departing from the invention. It is intended, therefore, by the appended to cover all such modifications and changes as may fall within the true spirit and scope of the invention.

What is claimed is:

1. A method for minimizing the epimerization of (+)-catechin to (+)-epicatechin and/or of (−)-epicatechin to (−)-catechin in a heat-processed food product having a moisture content of about 5% to about greater than 80% containing (+)-catechin and/or (−)-epicatechin comprises carrying out the heat processing at between about 37° C. and about 72° C. for from about 15 seconds to about 1.5 minutes while maintaining the pH of the product at between about 4 and about 6.

2. A method for maximizing the epimerization of (+)-catechin to (+)-epicatechin and/or of (−)-epicatechin to (−)-catechin in a heat-processed food product having a moisture content of about 5% to about 80% containing (+)-catechin or (−)-epicatechin comprises carrying out the heat processing at between about 100° C. and about 200° C. for from about 1 minute to about 30 minutes while maintaining the pH of the product at between about 7 and about 8.

3. The method of claim 1 or 2, wherein the food processing is carried out in an open food processor.

4. The method of claim 1 or 2, wherein the heat-processing is carried out in a closed food processor.

5. The method of claim 1 or 2, wherein heat-processing is carried out in the absence of oxygen.

6. The method of claim 5, wherein the heat-processing is carried out in the presence of an inert gas selected from the group consisting of nitrogen, argon, or helium.

7. The method of claim 1 or 2, wherein the heat processing is carried out under a vacuum.

8. The method of claim 1 or 2, wherein the heat processing is a pasteurization process or a sterilization process.

9. The method of claim 8, wherein the heating is carried out until the molar ratio of (−)-epicatechin to (−)-catechin is up to 1:2 and of (+)-epicatechin to (+)-catechin is up to 1:2.

10. The method of claim 1 or 2, wherein the food product is a fruit product, a vegetable product, a cereal product, a nut product, a spice product, or an edible botanical product.

11. The method of claim 10, wherein the fruit product is a blueberry, a cranberry, a blackberry, a raspberry, a strawberry, a bilberry fruit, a black currant, a cherry, a grape, an apple, an apricot, a kiwi, a mango, a peach, a pear, or a plum product; wherein the vegetable product is an Indian squash product; wherein the cereal product is a sorghum or a barley product; wherein the bean product is a black-eyed pea, a pinto bean, a small red bean, or a red kidney bean product; wherein the nut product is an almond, a cashew, a hazelnut, a pecan, a walnut, a pistachio, or a peanut product; or wherein the spice product is a curry or a cinnamon product; or wherein the edible botanical product is Chinese hawthorn, Acacia, Pterocarpus marsupium, Cassia Normane, rhubard, rhodiola, pine bark, willow bark, or Uncaria tomentosa.

12. The method of claim 1 or 2, wherein the food product is a cocoa product or a chocolate product.

13. The method of claim 12, wherein the cocoa product or food product contains (±) epicatechin and (±)-catechin.

14. An epimerized cocoa extract comprising a solution of water and optionally an organic solvent, which contains at least about 200 milligrams of cocoa polyphenols per gram of dried cocoa extract, wherein the cocoa polyphenols comprises (±) catechin, (±)-epicatechin, procyanidin dimers and trimers thereof.

15. The cocoa extract of claim 14, which is prepared by heating cocoa polyphenols dispersed in a water or an aqueous organic solvent at about 200° C. to about 0° C. for a time and at a pH sufficient to epimerize the (−)-epicatechin.

16. The cocoa extract of claim 15, which has been dried by removing the solvent and freeze-drying.

17. An epimerized cocoa powder containing at least about 25.0 milligrams of cocoa polyphenols per gram of defatted cocoa powder, wherein the cocoa polyphenols comprise (±) catechin, (±)-epicatechin, and procyanidin oligomers thereof.

18. A thermally-processed product containing cocoa solids, chocolate liquor, and/or a cocoa extract and containing at least about 6.0 milligrams of cocoa polyphenols per gram of the product, wherein the cocoa polyphenols comprise (±) catechin, (±)-epicatechin, and procyanidin oligomers thereof.
